(19) United States
(12) Patent Application Publication
(10) Pub. No.: US 2011/0087125 A1
(43) Pub. Date: Apr. 14, 2011

(54) SYSTEM AND METHOD FOR PAIN MONITORING AT THE POINT-OF-CARE

(76) Inventor: Elvir CAUSEVIC, Clayton, MO (US)

(21) Appl. No.: 12/576,521

(22) Filed: Oct. 9, 2009

(51) Int. Cl. A61B 5/0482; A61B 5/0484

(52) U.S. Cl. 600/544

(57) ABSTRACT

A method and apparatus for providing objective assessment of pain using a field portable device is described. The method includes placing an electrode set coupled to a handheld base unit on the subject's head, acquiring brain and/or peripheral nervous system electrical signals from the subject through the electrode set, processing the acquired brain electrical signals using a feature extraction algorithm stored in a memory of the base unit, classifying the processed signals into pain categories, determining an objective quantification of the pain level, and indicating the pain category and/or pain scale on the handheld base unit. The memory of the base unit stores a reference database for classification of the processed signals, or the base unit is configured to wirelessly access the reference database from a remote data storage unit.

This invention relates to the field of pain monitoring at a point-of-care setting, and more specifically, to a field-portable apparatus and method for detecting and measuring pain by recording and analyzing brain and peripheral nervous system electrical signals.

Pain is the most frequent complaint of patients seeking medical attention. It is, however, a difficult symptom to measure, and health care professionals most often rely on a patient's own report of pain, or “self-report” to assess their condition, using a pain intensity rating scale, such as the visual analog scale, pain faces scale, etc., which are currently considered the gold-standards for pain measurement. However, patient self-report may not always be accurate, as pain sensitivity and tolerance depend greatly on factors such as individual genotype, which are not taken into consideration by the standardized pain scales. An individual's response to a given pain stimulus is further influenced by prior pain experience, mental anxiety level etc., which render self-reported pain scores inherently subjective in nature. The lack of an objective measure for pain often results in disproportionate administration of analgesics, especially when the pain is of unknown etiology. Furthermore, reliance on patient self-report provides the opportunity for patients to manipulate the physician into over-administration or prescription of analgesic drugs.

Objective quantification of pain can be particularly beneficial for evaluating pain suffering in non-linguistic species, or where a patient is not communicative, such as when the patient is an infant, or the patient is semi-conscious or incoherent. It has already been shown in animal pain research studies that two independent approaches to the assessment of pain, spectral analysis of electroencephalogram (EEG) data and ethological analysis, when applied together can identify pain suffering in animals with much greater accuracy.

Similarly, human pain research can be greatly benefited if the self-reported pain scores are supplemented with an objective quantification of pain using brain and peripheral nervous system electrical signals. Brain imaging, using fMRI (Functional Magnetic Resonance Imaging), is currently used in pain research studies to understand nociceptive mechanisms, pain processing in the brain, pharmacological modulation of pain, etc. However, fMRI is expensive, requires trained personnel to operate and analyze results, and involves bulky and sensitive instrumentation, and therefore, not feasible for point-of-care settings. fMRI is also time-consuming and requires appointment scheduling, which makes it impractical for analysis of acute pain conditions.

Currently, electroencephalographic instrumentation (EEG) is used in surgical operations for monitoring anesthetization of patients, which includes evaluation of pain sensitivity. EEG and power spectral analyses of the recorded data are also used frequently in neuroscience research to study the neurogenic underpinnings of chronic pain. But application of EEG for pain measurement in a clinical setting is limited due to the requirement of experienced electroencephalographers to administer the test and interpret the recordings. U.S. Pat. No. 6,757,588 issued to Lange et al. discloses an objective pain measurement system based on EEG and power spectral analysis of the brain electrical signals of a subject. In the disclosed system, measurements quantifying a subject's pain level are made by processing the set of electrical activity measurements into a normalized signal, determining a level value for the normalized signal within a predetermined range of frequencies, and scaling the level value for the signal into an objective measurement of a subjective perception of pain. However, this system of pain measurement is crude in the sense that it does not facilitate the classification of pain signals, or enable the clinician to draw diagnostic inferences from the pain assessment.

The present disclosure describes a method and apparatus for detection, objective measurement and classification of pain using brain and peripheral nervous system electrical signals.

One aspect of the present disclosure includes a method of assessing pain experienced by a subject by acquiring electrical signals from at least one of the brain and peripheral nervous system using at least one electrode channel, extracting non-linear quantitative features from the acquired signals, classifying the extracted features into one or more pain states, and providing a quantitative value indicative of the level of pain perception.

Another aspect of the present disclosure includes a method of assessing pain experienced by a subject using evoked potentials generated in response to applied stimuli. In some exemplary embodiments, external sound stimuli is presented to the subject to generate auditory evoked potentials (i.e. ABR (auditory brainstem response), AMLR (auditory mid-latency responses), ALR (auditory late responses), P300, etc.). In other embodiments, small electrical signals are applied close to the nerves of the peripheral nervous system to elicit somatosensory evoked potentials (SSEP).

Yet another aspect of the present disclosure includes a portable pain monitoring device using Bx™ technology, for detecting the presence and/or severity of pain experienced by a subject, which includes a headset comprising at least one electrode for acquiring brain electrical signals, and a base unit comprising a processor configured to utilize one or more operating instructions stored in a memory to perform non-linear feature extraction from the brain electrical signals and classification of the extracted signal features into pain state.

It is to be understood that both the foregoing general description and the following detailed description are exemplary and explanatory only and are not restrictive of the invention, as claimed.

The accompanying drawings, which are incorporated in and constitute a part of this specification, illustrate embodiments of the invention and together with the description, serve to explain the principles of the various aspects of the invention.

### BRIEF DESCRIPTION OF THE DRAWINGS

FIG. 1 is a diagram illustrating an apparatus for recording and processing brain electrical signals to assess pain experienced by a subject, according to an exemplary embodiment consistent with the present disclosure.

FIG. 2 is a flowchart illustrating a method of pain assessment, according to an embodiment of the present disclosure;

FIG. 3 is flowchart showing the steps of providing an on-site pain assessment using a field-portable device, according to an embodiment of the present disclosure.

### DETAILED DESCRIPTION

Reference will now be made in detail to embodiments consistent with the present invention, examples of which are illustrated in the accompanying drawings. Wherever possible, the same reference numbers will be used throughout the drawings to refer to the same or like parts.

In an exemplary embodiment, data corresponding to brain electrical activity (including high frequency EEG not traditionally used in EEG assessment) is used to detect and quantify pain experienced by a subject. Analysis of numerous normal and pathological evaluations have demonstrated that brain electrical signals are highly sensitive to changes in normal brain function. In particular, brain electrical signals provide a direct indication of the activity of the cerebral cortex. Historically, the cerebral cortex was not believed to be an important structure in pain perception. But functional imaging techniques developed in the past decade have demonstrated that certain cortical structures, such as the anterior cingulate gyrus, play an important role in pain perception (Jones et al., “Functional imaging of pain perception”, 4, 2002, p. 329). These developments have greatly increased the role of brain electrical signal analysis as an indicator of the degree of pain perceived by a human subject or an animal.

In accordance with an exemplary embodiment of Bx™ technology, a subject's brain electrical activity is recorded using a varying number of electrodes located at standardized positions on the scalp and forehead, and the subject's brain electrical signals, in the presence and absence of pain, are assessed with reference to one or more databases. For example, collected normative data, indicative of normal brain electrical activity, is used to establish quantitative non-linear features of brain electrical activity, which clearly distinguish brain signals produced in the presence and absence of pain. This normative dataset includes brain activity data of a control group of population comprising of individuals similar to the subject in one or more aspects, such as age, gender, etc. The collected normative database employed by the inventor has been shown by others to be independent of racial background and to have extremely high test-retest reliability, specificity (low false positive rate) and sensitivity (low false negative rate). Further, the subject's brain electrical activity data may be compared to reference data showing brain activity of the control group in the presence of varying levels of pain. The database may further include individual baseline data, i.e., the subject's own brain electrical activity data in the absence of pain, or in the presence of a predetermined amount of pain, or a measurement prior to or during pain treatment.

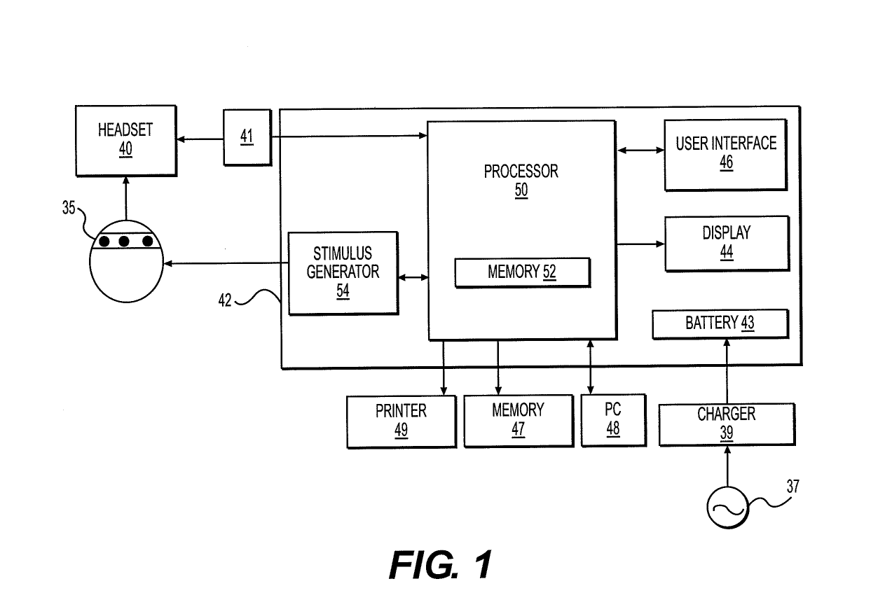

In accordance with certain embodiments of the present disclosure, FIG. 1 shows a pain monitoring instrument for acquiring and processing brain electrical signals using Bx™ technology, and providing an objective evaluation of the pain perceived by a subject. In an exemplary embodiment, the pain monitoring instrument is implemented as a portable device for point-of-care applications. This apparatus consists of a headset 40 which may be coupled to a base unit 42, which can be handheld, as illustrated in FIG. 1. Headset 40 may include a plurality of electrodes 35 to be attached to a patient's head to acquire brain electrical signals. The electrodes are configured for sensing both spontaneous brain activity as well as evoked potentials generated in response to applied stimuli, such as audio, tactile, or electrical stimuli. A simplest embodiment of the apparatus comprises a set of five anterior (frontal) electrodes: F1, F2, F7, F8 and Fz to be attached to a subject's forehead, and referenced to linked ears (A1+A2)/2 in accordance with the International 10/20 electrode placement system. The use of a limited number of electrodes enable rapid and repeatable placement of the electrodes on a subject, which in turn facilitates efficient, and more accurate, patient monitoring. Further, in one embodiment, the electrodes may be positioned on a low-cost, disposable platform, which can serve as a “one-size-fits-all” sensor. For example, electrodes 35 may be positioned on a head gear that is configured for easy and/or rapid placement on a patient, as further set forth in commonly assigned U.S. patent application Ser. No. 12/059,014, which is incorporated herein by reference in its entirety. Other electrode configurations may be utilized as and when required, as would be understood by those of ordinary skill in the art. Consistent with the present disclosure, the apparatus may also be applied for pain identification in animals, for instance in veterinary clinics, animal shelters, etc., which would naturally necessitate adjustments to the electrode set to accommodate the anatomical differences among different animals. Further, the headset may include a sound stimulus generator for eliciting auditory evoked potentials. Additionally, the apparatus may also include SSEP (somatosensory evoked potentials) electrical stimulators placed in standard locations in order to evoke a response from the nervous system.

In some embodiments, the headset 40 includes analog amplification channels connected to the electrodes, and an analog-to-digital converter (ADC) to digitize the acquired brain electrical signals prior to receipt by the base unit 42. The analog hardware in the base unit can include non-linear adaptive electronic systems, such as non-linear amplifiers, which would assist in the processing of high-frequency weak brain signals acquired in extremely noisy environments. The non-linear amplifier systems utilize either a non-linear scale for compression of the dynamic range (such as logarithmic) or a closed loop system to remove reference common mode noise from individual or groups of electrode channels from which the measurement is taken.

The base unit 42 may include a display 44, which can be a LCD screen, and can further have a user interface 46, which can be a touch screen user interface or a traditional keyboard-type interface. The interface 41 can act as a multi-channel input/output interface for the headset 40 and the handheld device 42, to facilitate bidirectional communication of signals to and from the processor 50, such that, for example, a command from the user entered through the user interface 46 can start the signal acquisition process of the headset 40. Interface 41 may include a permanently attached or detachable cable or wire, or may include a wireless transceiver, capable of wirelessly transmitting signals and receiving signals from the headset, or from an external device storing captured signals.

In an exemplary embodiment, noise artifacts are removed from the acquired signal in the signal processor 50, and the denoised signal is then processed to extract signal features and classify the extracted non-linear features, as per instructions loaded into memory 52, as set forth in commonly assigned U.S. patent application Ser. Nos. 11/195,001 and 12/041,106, which are incorporated herein by reference in their entirety. The memory 52 may further contain interactive instructions for using and operating the device to be displayed on the screen 44. The instructions may comprise an interactive feature-rich presentation including a multimedia recording providing audio/video instructions for operating the device, or alternatively simple text, displayed on the screen, illustrating step-by-step instructions for operating and using the device. The inclusion of interactive instructions with the device eliminates the need for extensive user training, allowing for deployment and use by persons other than medical professionals. The memory 52 may also contain a reference database, including collected population data or data indicative of the individual baseline. In an exemplary embodiment, a reference database may be accessed from a remote storage device via a wireless or a wired connection. Similarly, data collected from the subject by the pain monitoring instrument may be recorded in the database for future reference.

The result from the processor 50 maybe displayed on the display 44, or may be saved in external memory or data storage device 47, or may be displayed on a PC 48 connected to the base unit 42. In one embodiment, base unit 42 may contain a wireless power amplifier coupled to an antenna to transmit the results wirelessly to a remote network or PC 48 or the external memory 47 to store the results. In yet another embodiment, the results can be transmitted wirelessly or via a cable to a printer 49 that prints the results. Base unit 42 can also contain an internal rechargeable battery 43 that can be charged during or in between uses by battery charger 39 connected to an AC outlet 37. The battery can also be charged wirelessly through electromagnetic coupling by methods known in the prior art. Base unit 42 can also contain an antenna for receiving an RF emission from an external source.

In another embodiment, the processor 50 transmits a raw, unprocessed signal acquired from a subject to the computer 48. The computer can perform the denoising process, analyze the signal, extract nonlinear features, classify and output the results. The unprocessed brain electrical signals recorded from a subject may also be stored in an external memory device for future reference and/or additional signal processing.

In one exemplary embodiment consistent with the Bx™ technology, the headset 40 and the base unit 42 along with the charger 39 may come as a kit for field use or point-of-care applications. In yet another embodiment, both the headset 40 and the base unit 42 may be configured to reside on a common platform, such as a headband, to be attached to the subject's head. Furthermore, the processor 50 of the base unit, and the analog amplification channels and the ADC of the headset 40 may be configured to reside on a single integrated physical circuit, in accordance with the present disclosure and the Bx™ technology, and as further set forth in commonly assigned U.S. patent application Ser. No. 12/059,014, which is incorporated herein by reference in its entirety.

In another embodiment consistent with the present disclosure, the base unit 42 includes a stimulus generator 54 for applying pain stimuli to the subject to elicit evoked potentials. In some embodiments, the stimulus generator is included in the headset 40. Stimuli of varying levels of intensity may also be administered to the subjects using the stimulus generator 54 in order to extract signals correlating with different levels of pain perception. In some exemplary embodiments, external sound stimuli is presented to the subject to generate auditory evoked potentials (i.e. ABR (auditory brainstem response), AMLR (auditory mid-latency responses), ALR (auditory late responses), P300, etc.). In other embodiments, small electrical signals are applied close to the nerves of the peripheral nervous system to elicit somatosensory evoked potentials (SSEP). The processor 50 denoises and further processes both the spontaneous brain electrical signals as well as evoked potentials generated in response to the applied stimuli.

In certain embodiments, the pain monitoring instrument is used in a “calibration” mode in which the brain electrical activity of subjects are recorded while pain stimuli of varying intensities are administered to them. The subjects then rate their perceived pain using a conventional pain intensity rating scale, such as pain faces scale, etc., which allows cerebral activity to be correlated to the stimulus intensities for producing different pain levels, and more importantly, it enables correlation of brain electrical activity to subjective rating reflecting individual pain experience.

The pain monitoring instrument, developed in accordance with the Bx™ technology, is designed for near-patient testing in hospitals, doctors' offices, ambulatory setting, etc., in combination with the traditional pain intensity rating scales, such as the visual analog scale, pain faces scale, etc. The key objective of point-of-care pain evaluation is to generate fast triage results, especially where there is limited access to well-trained medical technicians, so that appropriate treatment can be quickly provided, leading to an improved overall clinical outcome. For example, the pain monitoring instrument may be used by an EMT, an ER nurse, or any other medical professional to quantify the pain experienced by a patient, which in turn will help to determine an appropriate pain management strategy, or assist in making a preliminary diagnosis. In addition, the pain monitoring instrument is designed to be field-portable, that is, it can be used in locations far removed from a full-service clinic—for example, in remote battlefield situations where traditional military healthcare system is not available, resource-poor environments where there is limited access to electricity and trained medical professionals, or during sporting events for indentifying if an injured athlete should be transported for emergency treatment. The pain monitoring instrument may be utilized for many different clinical and non-clinical applications including, but not limited to, management of acute and/or chronic pain, prescription of analgesics to patients, regulation of analgesic drug delivery to patients using Patient Controlled Analgesic (PCA) devices, rehabilitation treatment, etc. Additionally, the pain monitoring system may be used for closed-loop control of analgesia in subjects experiencing persistent severe pain. The pain monitoring instrument may be used as a feedback controller to monitor the level of pain and regulate the dosage and rate of delivery of pain medication to the subject via devices and methods known in the art. Similarly, the pain monitoring instrument may be used as a feedback controller for intraoperative closed-loop control of analgesia. In such as system, the output from the pain monitoring instrument may be used to directly control the infusion rate of the analgesic delivery pump. The pain monitoring device may also be used to predict responsiveness to medication from a premedication baseline. The pain monitoring instrument, developed in accordance with the Bx™ technology, may also be used for pain testing in animal subjects in veterinary clinics, animal shelters, etc.

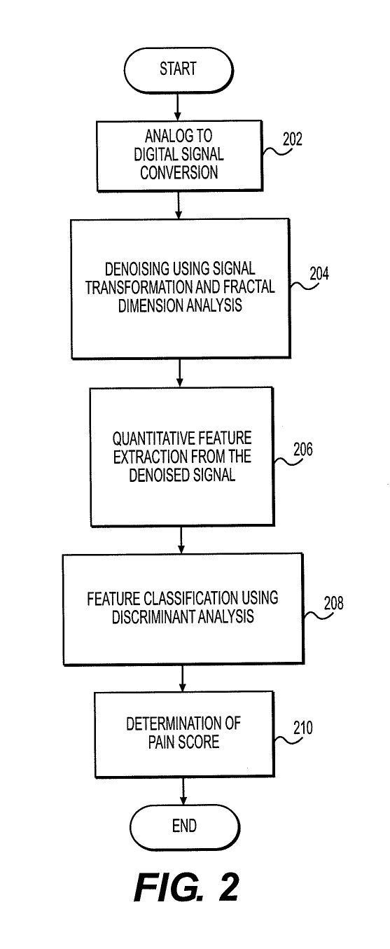

In an embodiment consistent with the present disclosure, the brain electrical signals are processed to remove noise, processed to extract features, and further processed to classify the extracted features, according to the method illustrated in FIG. 2. More specifically, memory 52 can contain instructions that are executed by the processor 50 to extract quantitative features from the signal, classify the extracted features into pain states using a reference database, and derive an objective pain score indicative of the level of pain perception.

Consistent with the present disclosure and the Bx™ technology, processing the acquired signals comprise digitizing the recorded analog brain electrical signals (step 202), and performing an algorithm for automatic artifact identification and removal from the acquired signals (step 204) using a signal processing method described in commonly-assigned U.S. patent application Ser. No. 12/106,699, which is incorporated herein by reference in its entirety. In one embodiment, the artifact identification and rejection algorithm follows the following steps:
a. Transforming the signal into a plurality of signal components;
b. Computing fractal dimension of the components;
c. Identifying noise components based on their fractal dimension;
d. Automatically attenuating the identified noise components;
e. Reconstructing a denoised signal using inverse transform.

In some exemplary embodiments, the input analog brain electrical signal is at first digitized and then deconstructed into its constitutive coefficients using a linear or non-linear signal transformation method, such as Fast Fourier Transform, Independent Component Analysis (ICA)-based transform, wavelet transform, wavelet packet transform etc. The fractal dimensions of the coefficients are then calculated in the transform domain, and the coefficients that have a fractal dimension higher than a threshold value are attenuated. The intact and re-scaled coefficients are then remixed using an inverse signal transform to generate a denoised signal, which is then further processed to extract signal features and classify the extracted features.

The next step of signal processing comprises extracting quantitative non-linear features from the denoised signal (step 206). In certain embodiments, linear signal features are also extracted, and subsequently combined with the non-linear features for further signal processing. The feature extraction algorithm takes as input a number of “artifact-free” or “denoised” epochs having a temporal length of 2.56 seconds, which corresponds to 256 samples for data sampled at 100 Hz (or similar proportion for other sampling frequencies). In an exemplary embodiment, processor 50 is configured to perform a linear feature extraction algorithm based on Fast Fourier Transform and power spectral analysis, according to a method disclosed in commonly-assigned U.S. patent application Ser. Nos. 11/195,001 and 12/041,106, which are incorporated herein by reference in its entirety. In short, the algorithm performs feature selection using Fourier transform of narrow frequency bands and calculating the power at each frequency band. The frequency composition can be analyzed by dividing the signal into the traditional frequency bands: delta (1.5-3.5 Hz), theta (3.5-7.5 Hz), alpha (7.5-12.5 Hz), beta (12.5-25 Hz), and gamma (25-50 Hz). Higher frequencies, up to and beyond 1000 Hz may also be used, even though they are not traditionally used for EEG analysis. The reason for using these high frequencies is that it is believed that the high frequency signals may be generated by structures deeper in the brain than those generating the low frequency signals. This is particularly useful for the pain application, since the pain regions may correspond to the regions in the brain where the high frequency signals are generated. Using traditional quantitative EEG (qEEG), univariate features are computed by calculating the absolute and relative power for each of the electrodes or between a pair of electrodes within selected frequency bands, and the asymmetry and coherence relationships among these spectral measurements within and between the sets of electrodes. The processor 50 may also be configured to compute multivariate features, which are linear or non-linear functions of groups of the univariate features involving two or more electrodes or multiple frequency bands. The computed measures are normalized by performing age-regression and Z-transformation to obtain features (Z-scores) for discriminant analysis. Non-linear features are computed across the entire range of non-linear transforms, including but not limited to wavelets, wavelet packets, fractals, etc.

In another embodiment, processor 50 is configured to perform a non-linear feature extraction algorithm based on wavelet transforms, such as Discrete Wavelet Transform (DWT) or Complex Wavelet Transforms (CWT). In yet another embodiment, processor 51 is configured to perform feature extraction using non-linear signal transform methods, such as wavelet packet transform. The features extracted by this method are referred to as Local Discriminant Basis (LDB) features. The LDB algorithm defines a set of features that are optimized for the statistical discrimination between different classes of signals. The computation of these features begin with the calculation of power spectral densities over a set of epochs for each electrode channel. For each subject, the algorithm produces one power spectrum for each channel, and quotients of the power spectra for each pair of channels are then calculated. Thus, for a 5 channel system, a set of 15 power spectra per subject is produced, which allows for the calculation of 15 distinct bases (sets of LDB vectors). The LDB features are then obtained by calculating a wavelet packet table for each power spectrum using the Haar or another wavelet function. The function is applied to both the low pass and the high pass sub-bands, which generates a tree structure providing many possible wavelet packet bases, and accordingly, signals are decomposed into a time-frequency dictionary. The selection of the best basis can be done manually or algorithmically.

In another embodiment consistent with the present disclosure, diffusion geometric analysis is used to extract non-linear features according to a method disclosed in commonly-assigned U.S. patent application Ser. No. 12/105,439, which is incorporated herein by reference in its entirety. The recorded brain electrical activity dataset is at first organized into a plurality of digital documents, each including a time window of temporal features of the measurement of each electrode. The affinity between the documents may then be computed using an appropriate affinity matrix A. An affinity matrix A, between a document at time i and a document at time j may be defined as

$${A}_{i , j} = \frac{{e}^{\frac{- {‖ v ( i ) - v ( j ) ‖}^{2}}{ɛ}}}{w ( i ) w ( j )} ,$$

wherein ε is a threshold parameter, w(i) is a weighting function at time i, and w(j) is the weighting function at time j, and the weighting functions are selected such that A is Markov in i and j. Next, the eigenvectors of the affinity matrix A are determined, and the eigenvectors are used to construct a Euclidean space representing the diffusion geometry of the dataset comprising at least a plurality of diffusion coordinates. If the first three eigenvectors are used, we obtain an embedding into three dimensional Euclidean space, where the diffusion metric (relational inference) is isometrically converted to corresponding Euclidean distance. The dataset can then be classified based on the metrics provided by the diffusion geometry, and a pain state can be identified based on the classification. The dataset may be classified by a number of different criteria. In one embodiment, predetermined portions of the diffusion coordinates space may be partitioned into partitions corresponding to particular pain states. In another embodiment, computing a diffusion geometry on the plurality of digital documents may result in clusters in the multi-scale structure. The clusters may represent specific classifications, depending on the metrics used to initialize the cluster. Each metric in the multi-scale structure corresponds to one of the diffusion distances of the plurality of digital documents. A cluster is at first initialized based on one metric, and then hierarchically aggregated based on a different metric from the multiplicity of metrics corresponding to the diffusion distances. In yet another embodiment, classifying the neurological state comprises the step of comparing a present dataset to another stored dataset based on diffusion geometry associated with each dataset.

Referring again to FIG. 2, the extracted signal features (such as the diffusion geometry features, Local Discriminant Basis features, etc.) may also be classified into pain states or scales using a classification algorithm, such as Linear Discriminant Analysis (LDA) (step 208). All the extracted features are age-regressed and z-transformed for discriminant analysis. The LDA optimally combines the features (Z-scores) into a discriminant score that possesses the maximum discriminating power. In one embodiment, the discriminant analysis used is a two category classifier (also called “dichotomizer”) which assigns for each given subject a discriminant score between 1 and 100. For example, a score “lower than 50” indicates that the subject is more likely to belong to pain state A than to pain state B, and vice versa. Examples of different classification classes include, but is not limited to, normal (absence of pain) vs. abnormal (presence of pain), mild vs. severe pain, acute vs. chronic, etc. The discriminant scores, S_{A} and S_{B} corresponding to classes A and B, are computed for any subject with the following Fisher LDA formulas:

S_{A}=100·G(1)/(G(1)+G(2)), S_{B}=100·G(2)/(G(1)+G(2))

G(1)=exp(Z·W_{A}+C_{A}), G(2)=exp(Z·W_{B}+C_{B})

where Z denote the set of age-regressed z-transformed features (discriminants) computed for any subject. W_{A} and W_{B} denote two weight vectors that are derived from a reference database, and C_{A} and C_{B} are two constants which are commonly called bias or threshold weights. The weights are pre-selected using a training routine such that they result in the ‘best’ separation between the classes. The weights for the different monopolar and/or bipolar univariate and multivariate features may be estimated from a stored population reference database, such as a database comprising of population normative data indicative of brain electrical activity of a first plurality of individuals in the absence of pain, population reference data indicative of brain electrical activity of a second plurality of individuals generated in response to applied pain stimuli, or subjective population reference data indicative of brain electrical activity of a third plurality of individuals reporting different levels of pain vis-à-vis a subjective pain scale (e.g. the pain faces scale). Similarly, the weights may be selected from a database of the subjects own brain electrical activity data generated in the absence or presence of pain.

The discriminant scores can be further converted to probabilities of correct and incorrect classification using Receiver Operating Characteristics (ROC) curves, if the true classification information (diagnosis) for a sample group is available. The ROC curves indicate the threshold, sensitivity, specificity, positive predictive value (probability that the disease is present when the classification result is positive), and negative predictive value (probability that the disease is absent when the classification result is negative) which can be expected from a particular algorithm/classifier. The output of the two-state discriminant analysis, as described above, is a number that can take any value between 0 and 100. Once a critical value (or threshold) T is selected, the output of the test becomes binary, and sensitivity and specificity for that particular threshold can be calculated. The ROC is the curve through the set of points: {(1-specificity(T), sensitivity(T))}, which is obtained by varying the value of the critical value T between 0 and 1. ROC curves are therefore an illustration of the achievable statistical performance of a classifier, depending on the selected critical value. Any other type of classifier (for example, Partial Least Squares classifier, quadratic classifier, etc.), may also be used in place of LDA, and the data is not sensitive to the choice of classifier. In some embodiments, non-linear classifiers are used. A combination of classifiers can be used in various voting strategies as well (choosing the results of any 3 of 5, 2 of 3, etc).

In addition to classifying the data into pain intensity categories and/or diagnostic categories, the discriminant score obtained from the classification can be used to indicate quantitative pain scores (step 210), which would provide a numerical indication of the level of pain experienced by a subject (such as the traditionally used pain scales with 1-10 rating, or pain faces scale). The pain scores can be further used to generate an objective Pain Index by calibrating the numerical scores to scales of pain ranging from absent, sensation of discomfort, mild pain, moderate pain, or severe pain. This Pain Index may be used in combination with the subjective pain scales based on patient self-report, to evaluate the pain suffering of the patient.

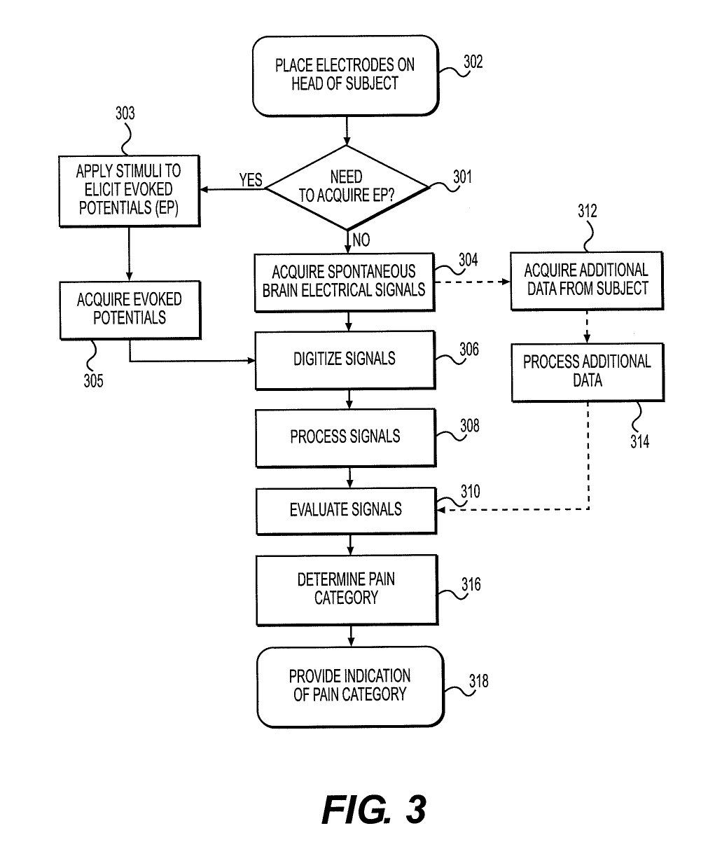

FIG. 3 shows a flowchart diagramming the steps of providing an on-site pain assessment using a field portable device, in accordance with an embodiment of the present disclosure, and will be described in conjunction with FIG. 1 to illustrate the method. The electrodes 35 are first placed on the head of the subject (step 302). The handheld base unit 42 is then powered on using power supplied by the battery 43. The processor 50 executes instructions stored in the memory 52 to display instructions for operating the device. An user can use the user interface 46 to enter a command to start signal acquisition. If the user determines that evoked potentials may also have to be recorded (step 301), he may initiate stimulus generator 54 and apply stimuli to elicit evoked potentials (step 303). Brain electrical signals, which may include at least one of the spontaneous and evoked potentials, are acquired from electrodes 35 (steps 304 and/or 305), the signal is digitized (step 306), and then passed to the processor 50 for signal processing (i.e. denoising, feature extraction and classification) (step 308).

In some embodiments, a user may also acquire additional data from the subject (step 312). Such additional data may include video data showing activity of any body system responsive to pain, such as the subject's facial expressions, eye movement, contraction of muscles of the forehead, sweating, tearing of the eyes, etc., and audio data including the subject's responses to questions about his pain condition. At the time of enrollment, information may also be collected from the subject using a symptom checklist, and a standard pain scale. As known in the prior art, the symptom checklist can be used to rate the severity of pain symptom for the current as well as previously sustained injuries or physiological conditions. This information may be entered into the processor 50 by the user, and can be used in conjunction with the processed brain electrical signals to provide an evaluation of the acquired signals (step 310) and determine the presence and/or the intensity of pain, or the diagnostic category of the pain (step 316). Following the determination of the pain category, processor 50 executes instructions to provide an indication of the pain diagnosis (step 318) to be displayed by display electronics 44. The indication may comprise a color-coded indication, or a simple message displayed on screen indicating a pain scale.

Embodiments consistent with the present disclosure, using advanced signal processing algorithms and stored data of the brain electrical signals of thousands of subjects having different pain indications, may provide a rapid and accurate assessment of the pain experienced by a subject. Moreover, the advanced signal processing algorithms may be executed by a processor capable of integration in a portable handheld device. The portable handheld device used with a reduced electrode set allows for a rapid, on-site solution for pain measurement, and determining an appropriate course of treatment at the early stage of an injury or other pathophysiological condition resulting in pain. Also, the entire signal processing hardware and algorithms can be embedded on a chip for use as a standalone device, or as part of the headset or an implanted device. Further, the pain measurement algorithm can be programmed into already-implanted pain stimulators in the brain or the peripheral nervous system.

Other embodiments of the invention will be apparent to those skilled in the art from consideration of the specification and practice of the invention disclosed herein. It is intended that the specification and examples be considered as exemplary only, with a true scope and spirit of the invention being indicated by the following claims.

What is claimed is:

1. A method of assessing pain experienced by a subject comprising the steps of:
providing at least one electrode channel;
acquiring electrical signals from at least one of the brain and the peripheral nervous system of the subject using the at least one electrode channel;
providing at least one processor, the at least one processor performing the steps of:
extracting non-linear quantitative features from the acquired signals;
classifying the extracted features into one or more pain states; and
providing a quantitative value indicative of the level of pain perception.

2. The method of claim 1, wherein the step of classifying the extracted features is performed using a reference database stored in a memory device.

3. The method of claim 2, wherein the reference database comprises brain electrical activity data from a plurality of individuals in the presence or absence of pain.

4. The method of claim 3, wherein the reference database comprises the subject's own brain electrical activity data in the presence or absence of pain.

5. The method of claim 1, wherein the non-linear features are combined with linear signal features.

6. The method of claim 1, wherein the quantitative value is used to generate an objective pain index.

7. The method of claim 1, further comprising the steps of:
amplifying the acquired brain electrical signals linearly or non-linearly;
digitizing the amplified brain electrical signals with the at least one processor; and
denoising the digitized signals.

8. The method of claim 1, further comprising the step of stimulating the subject with a stimulus generator to obtain evoked potentials.

9. The method of claim 1, wherein the electrical signals from the brain comprises spontaneous electrical activity.

10. The method of claim 1, wherein the electrical signals from the brain comprises spontaneous high-frequency electrical activity.

11. The method of claim 1, wherein the electrical signals from the brain comprises evoked potentials.

12. The method of claim 1, wherein the electrical signals from the brain comprises spontaneous electrical activity and evoked potentials.

13. The method of claim 1, wherein the step of feature extraction is performed using diffusion geometric analysis.

14. The method of claim 1, wherein the step of feature extraction is performed using wavelet packet transformation.

15. The method of claim 1, wherein the step of classifying the extracted features is performed using linear discriminant analysis.

16. The method of claim 1, wherein the step of classifying the extracted features is performed using a non-linear classifier.

17. The method of claim 1, wherein the step of classifying the extracted features is performed using voting strategy to combine multiple classifiers.

18. The method of claim 1, wherein the assessment of pain experienced by a subject is performed using a portable, handheld device.

19. The method of claim 18, wherein the device can be operated in a calibration mode to correlate brain electrical activity to stimulus levels and to subjective ratings reflecting individual pain experience based on either externally derived pain levels or by providing a pre-set pain stimulus.

20. The method of claim 18, wherein the device can be operated as a feedback controller for closed-loop administration of analgesic drugs to a subject.

21. The method of claim 18, wherein the device can be used to predict responsiveness to medication from a pre-medication baseline.

22. The method of claim 18, wherein the device can be used to predict medication for a particular type of pain and its severity.

23. A device for assessing pain experienced by a subject, comprising:
a headset comprising at least one electrode for acquiring brain electrical signals;
a base unit; wherein
the base unit comprises a processor configured to utilize one or more operating instructions stored in a memory to perform non-linear feature extraction from the brain electrical signal and classification of the extracted signal features.

24. The device of claim 23, wherein the processor is configured to output an objective measurement of pain.

25. The device of claim 23, wherein the processor is configured to output a result indicating a pain category.

26. The device of claim 23, further comprising a display wherein a result of one or more operations performed by the processor is displayed.

27. The device of claim 23, wherein the display is operatively connected to the processor; and
wherein the display can be integrated into the base unit, or can be external to the base unit.

28. The device of claim 25, wherein the base unit communicates wirelessly with an external display.

29. The device of claim 23, wherein the headset communicates wirelessly with the base unit.

30. The device of claim 23, wherein the headset comprises non-linear, adaptive electronic systems.

31. The device of claim 23, wherein the base unit comprises a stimulus generator to apply stimuli to the subject.

32. The device of claim 29, wherein the processor is configured to process spontaneous brain electrical signals and evoked potentials generated in response to the applied stimuli.

33. The device of claim 23, wherein the headset and the base unit are configured to reside on a single platform to be connected to the subject.

34. The device of claim 23, wherein the memory stores reference data for classification of the extracted signal features.

35. The device of claim 34, wherein the reference data is stored in an external data storage device.

36. The device of claim 35, wherein the data from the external storage device is accessed wirelessly by the processor.

37. The device of claim 23, wherein a result of one of more operations performed by the processor is stored in the memory.

38. The device of claim 37, wherein the result is stored in an external storage device.

39. The device of claim 23, wherein the brain electrical signals recorded from a subject are stored in an external storage device.

40. The device of claim 23, further comprising at least a second electrode to acquire electrical signals from the peripheral nervous system of the subject.
